(19) United States
(12) Patent Application Publication
(10) Pub. No.: US 2026/0301899 A1
(43) Pub. Date: Oct. 1, 2026

(54) MEDICAL INFORMATION PROCESSING APPARATUS, METHOD, AND STORAGE MEDIUM

(71) Applicant: NEC Corporation, Tokyo (JP)

(72) Inventors: Kei SHIBUYA, Tokyo (JP); Masanori TSUJIKAWA, Tokyo (JP)

(73) Assignee: NEC Corporation, Tokyo (JP)

(21) Appl. No.: 19/565,999

(22) Filed: Mar. 13, 2026

(30) Foreign Application Priority Data
Mar. 26, 2025 (JP) 2025-052189

(51) Int. Cl. G16H 15/00; G16H 10/60

(52) U.S. Cl. CPC G16H 15/00; G16H 10/60

(57) ABSTRACT

A medical information processing apparatus 1X includes an acquisition means 14X, a classification means 15X, a summary generation means 17X, and a summary combining means 18X. The acquisition means 14X acquires medical records of a patient. The classification means 15X classifies the medical records for each of categories related to a daily life state. The summary generation means 17X generates, for each of the categories, a summary of the medical records classified for each of the categories. The summary combining means 18X generates a summary document obtained by combining the summaries of the categories.

### INCORPORATION BY REFERENCE

This application is based upon and claims the benefit of priority from Japanese Patent Application No. 2025-052189, filed on Mar. 26, 2025, the disclosure of which is incorporated herein in its entirety by reference.

### TECHNICAL FIELD

The present disclosure relates to a technical field of a medical information processing apparatus, a method, and a storage medium for processing medical information.

### BACKGROUND

Among various types of work performed by medical workers, work of recording a medical practice performed by a medical worker on a patient is essential for sharing patient information with a related medical worker, proving that an objectively appropriate medical practice has been performed, and the like. On the other hand, the work related to the medical record has a large burden on the medical worker. As a technology related to generation of a summary document based on the medical record, Patent Literature 1 discloses a technology of generating a summary from medical records by a medical worker.

### Citation List

### Patent Literature

Patent Literature 1: JP 2020-38602 A

### SUMMARY

At the time of discharge of a patient or the like, it is necessary for a nurse to prepare a summary document, such as a nursing summary, regarding a daily life state of the patient after the discharge. In a case where such a summary document is generated by manual work, it takes time to retrospectively read daily records during hospitalization and search for information. On the other hand, automatic generation of such a summary document regarding the daily life state by artificial intelligence (AI) has a different point of description from general generation of a summary document of medical records such as a discharge summary created by a doctor, and thus needs to be devised.

In view of the above-described problems, an object of the present disclosure is to provide a medical information processing apparatus, a method, and a program capable of generating a summary document of medical records regarding a daily life state.

In an example aspect of the present disclosure, there is provided a medical information processing apparatus including: an acquisition means for acquiring medical records of a patient; a classification means for classifying the medical records for each of categories related to a daily life state; a summary generation means for generating, for each of the categories, a summary of the medical records classified for each of the categories; and a summary combining means for generating a summary document obtained by combining the summaries of the categories.

In an example aspect of the present disclosure, there is provided a method executed by a computer, including: acquiring medical records of a patient; classifying the medical records for each of categories related to a daily life state; generating, for each of the categories, a summary of the medical records classified for each of the categories; and generating a summary document obtained by combining the summaries of the categories.

In an example aspect of the present disclosure, there is provided a program executed by a computer, the program causing the computer to: acquire medical records of a patient; classify the medical records for each of categories related to a daily life state; generate, for each of the categories, a summary of the medical records classified for each of the categories; and generate a summary document obtained by combining the summaries of the categories.

An example advantage according to the present disclosure is to generate a summary document of medical records regarding a daily life state.

### BRIEF DESCRIPTION OF THE DRAWINGS

FIG. 1 illustrates a schematic configuration of a medical information processing system.

FIG. 2 illustrates a hardware configuration of a medical information processing apparatus.

FIG. 3 is an example of functional blocks of a processor of the medical information processing apparatus.

FIG. 4 is a first display example of a summary document.

FIG. 5 is an enlarged view in which annotations are added to the summary document displayed in an AI-generated document display field.

FIG. 6 is a second display example of the summary document.

FIG. 7 is an example of a flowchart executed by the medical information processing apparatus.

FIG. 8 illustrates a schematic configuration of a medical information processing system.

FIG. 9 is a block diagram of a medical information processing apparatus.

FIG. 10 is an example of a flowchart illustrating a processing procedure executed by the medical information processing apparatus.

### EXAMPLE EMBODIMENT

Hereinafter, example embodiments of a medical information processing apparatus, a method, and a storage medium will be described with reference to the drawings.

### First Example Embodiment

### (1) System Configuration

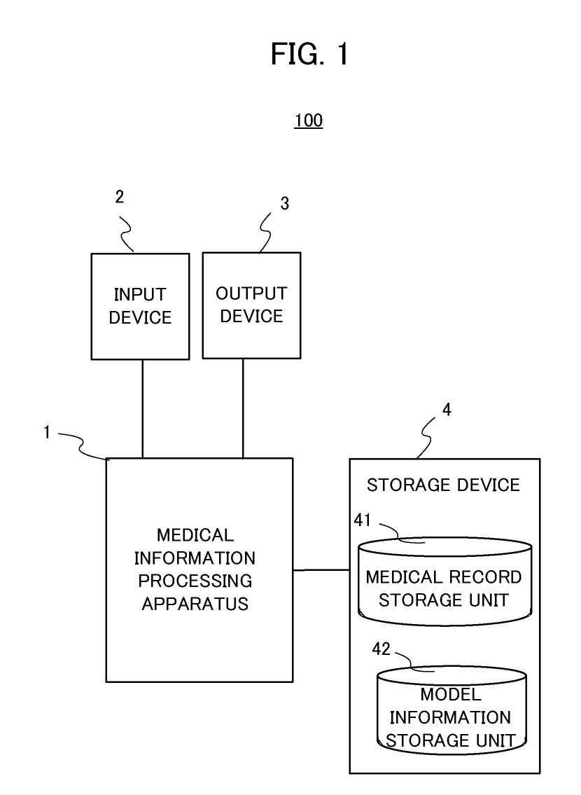

FIG. 1 illustrates a schematic configuration of a medical information processing system 100. The medical information processing system 100 generates a summary document of medical records regarding a daily life state of a designated patient.

Hereinafter, the “medical records” refer to various records created in a process of medical treatment for states (a physical condition, a condition of a disease, treatment to be applied, and the like) of the patient, and includes a medical record (clinical record), a prescription, an operation record, a nursing record, an examination observation record, an X-ray photograph, a referral, and the like. Hereinafter, unless otherwise stated, the medical records are assumed to refer to electronic data in which the above-described various records are electronically stored. In addition, the summary document generated by the medical information processing system 100 from the medical records is a summary of sentences indicating the daily life state included in the medical records, and may be used for, for example, a document (memo) describing a treatment progress, a discharge summary, a nursing summary (nursing contact form), and the like.

The medical information processing system 100 mainly includes a medical information processing apparatus 1, an input device 2, an output device 3, and a storage device 4.

The medical information processing apparatus 1 performs processing related to generation of a summary document of the medical records regarding the daily life state of the designated patient. The medical information processing apparatus 1 performs data communication with the input device 2, the output device 3, and the storage device 4 via a communication network or by wireless or wired direct communication. The medical information processing apparatus 1 detects, for example, a request (also referred to as a “summary generation request”) for generating the summary document regarding the daily life state based on an input signal supplied from the input device 2 and the like. The summary generation request includes, for example, information designating the patient, information instructing the start of the processing of generating the summary document, and the like. The medical information processing apparatus 1 also generates an output signal, and supplies the generated output signal to the output device 3. The output signal in this case is, for example, display information (which may include audio information) for receiving an input necessary for generating the summary document or a correction input of the generated summary document. The medical information processing apparatus 1 may cause the generated information regarding the summary document to be stored in the storage device 4 or to be transmitted to an external device (not illustrated). The generated summary document may be used, for example, for a referral, a medical certificate, and a discharge summary, which are created by medical workers, and other documents that require description of the summary document of the medical records regarding the daily life state.

The input device 2 is an interface that receives a manual input (external input) of information by a user. The input device 2 may be, for example, various user input interfaces such as a touch panel, a button, a keyboard, a mouse, and an audio input device. The input device 2 supplies a generated input signal to the medical information processing apparatus 1. The output device 3 outputs predetermined information based on the output signal supplied from the medical information processing apparatus 1. The output device 3 is, for example, a display, a projector, a speaker, or a printer.

The storage device 4 is a memory that stores various types of information required for processing executed by the medical information processing apparatus 1. In terms of functions, the storage device 4 mainly includes a medical record storage unit 41 that stores the medical records of the patients and a model information storage unit 42 that stores model information necessary for execution of a machine learning model used by the medical information processing apparatus 1.

The medical record storage unit 41 stores the medical records of the patients. Each record of the medical records includes, for example, identification information of the medical record, information on the date and time when the medical record is created (or medical treatment is performed), identification information of the patient, and content of the medical record (for example, a clinical record). Note that a medical record regarding a daily life state of a patient is generated by, for example, a nurse or other medical staff inputting a daily state of the patient using the input device 2 or the like, and the generated medical record is stored in the medical record storage unit 41 in association with identification information of the patient, date and time information, and the like. In addition, basic information of each of the patients is stored in the storage device 4 in association with a patient ID. The basic information of the patient includes, for example, attributes (age, occupation, medical history, and the like) of the patient, identification information of a hospital and a clinical department where the patient receives medical treatment, and the like.

The model information stored in the model information storage unit 42 includes, for example, model information (configuration information) for configuring a machine-learned large language model (LLM), model information for configuring any natural language understanding model used for natural language processing, and the like. Each piece of the model information includes, for example, various parameters of a machine-learned deep learning model such as a layer structure, a neuron structure of each layer, the number of filters and sizes of the filters in each layer, and weights of elements of the filters.

Here, a definition of a large language model or a language model will be described. The language model is a machine learning model that has learned relationships between words in sentences and generates, from an object character string, a related character string related to the object character string. By using the language model that has learned sentences in various contexts, it is possible to generate the related character string having appropriate content related to the object character string. For example, a case where the language model is used in question answering will be described. The language model receives input of a question “What kind of country is Japan?” as a target character string. At this time, the received question is also referred to as a “prompt”. The language model generates a character string such as “Japan is an island country in the Northern Hemisphere ...” as an answer to the question. A training method of the language model is not particularly limited, but as an example, the language model may be trained in such a way as to output at least one sentence including an input character string. As a specific example, the language model may be a generative pre-trained transformer (GPT) that outputs a sentence including an input character string by predicting a character string having a high probability of following the input character string, or ChatGPT based on the GPT. In addition, for example, text-to-text transfer transformer (T5), bidirectional encoder representations from transformers (BERT), robustly optimized BERT approach (RoBERTa), efficiently learning an encoder that classifies token replacements accurately (ELECTRA), and the like are also the language models.

The storage device 4 may be an external storage device such as a hard disk connected to or incorporated in the medical information processing apparatus 1, or may be a storage medium such as a flash memory. At least a part of the information stored in the storage device 4 may be stored by the medical information processing apparatus 1. The storage device 4 may be a server device that performs data communication with the medical information processing apparatus 1. The storage device 4 may include a plurality of devices. In this case, the server device may execute the machine learning model (language model) based on the model information, instead of the medical information processing apparatus 1. In this case, the server device executes the machine learning model in a case where information (for example, a prompt) necessary for execution of the machine learning model and the like are received from the medical information processing apparatus 1, and transmits information generated by the machine learning model to the medical information processing apparatus 1.

Note that, a configuration of the medical information processing system 100 illustrated in FIG. 1 is an example, and various changes may be made to the configuration. For example, the input device 2 and the output device 3 may be integrally configured. In this case, the input device 2 and the output device 3 may be configured as a tablet terminal (including a smartphone) integrated with or separated from the medical information processing apparatus 1. The medical information processing apparatus 1 may include a plurality of devices. In this case, the plurality of devices forming the medical information processing apparatus 1 exchange information necessary for executing processing allocated in advance between the plurality of devices. In this case, the medical information processing apparatus 1 functions as a system.

### (2) Hardware Configuration

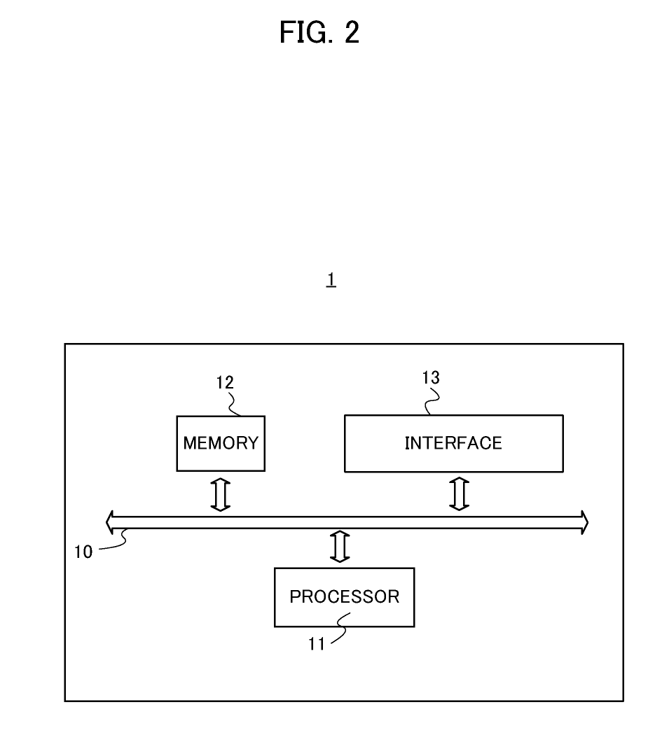

FIG. 2 illustrates a hardware configuration of the medical information processing apparatus 1. The medical information processing apparatus 1 includes a processor 11, a memory 12, and an interface 13 as hardware. The processor 11, the memory 12, and the interface 13 are connected via a data bus 10.

The processor 11 functions as a controller (arithmetic unit) that controls the entire medical information processing apparatus 1 by executing a program stored in the memory 12. The processor 11 is, for example, a processor such as a central processing unit (CPU), a graphics processing unit (GPU), and a tensor processing unit (TPU). The processor 11 may be configured by a plurality of processors. The processor 11 is an example of a computer.

The memory 12 is configured by various types of volatile memories and nonvolatile memories such as a random access memory (RAM), a read only memory (ROM), and a flash memory. In addition, the memory 12 stores a program for executing processing executed by the medical information processing apparatus 1. Note that a part of information stored in the memory 12 may be stored by one or a plurality of external storage devices capable of communicating with the medical information processing apparatus 1, or may be stored by a storage medium detachable from the medical information processing apparatus 1.

The interface 13 is one or more interfaces for electrically connecting the medical information processing apparatus 1 and other devices. These interfaces may be a wireless interface such as a network adapter for wirelessly performing transmission and reception of data to and from other devices, or may be a hardware interface for connecting to other devices by a cable or the like.

Note that the hardware configuration of the medical information processing apparatus 1 is not limited to the configuration illustrated in FIG. 2. For example, the medical information processing apparatus 1 may include at least one of the input device 2 or the output device 3.

### (2) Summary Document Generation Processing

Next, details of summary document generation processing, which is processing of generating a summary document of medical records regarding a daily life state of a patient, executed by the medical information processing apparatus 1 will be described. Schematically, when detecting a summary generation request, the medical information processing apparatus 1 summarizes medical records classified into categories for each of the categories, and generates a summary document obtained by combining the summaries of the respective categories. As a result, the medical information processing apparatus 1 generates an easily readable summary document in which sentences are collected for each category.

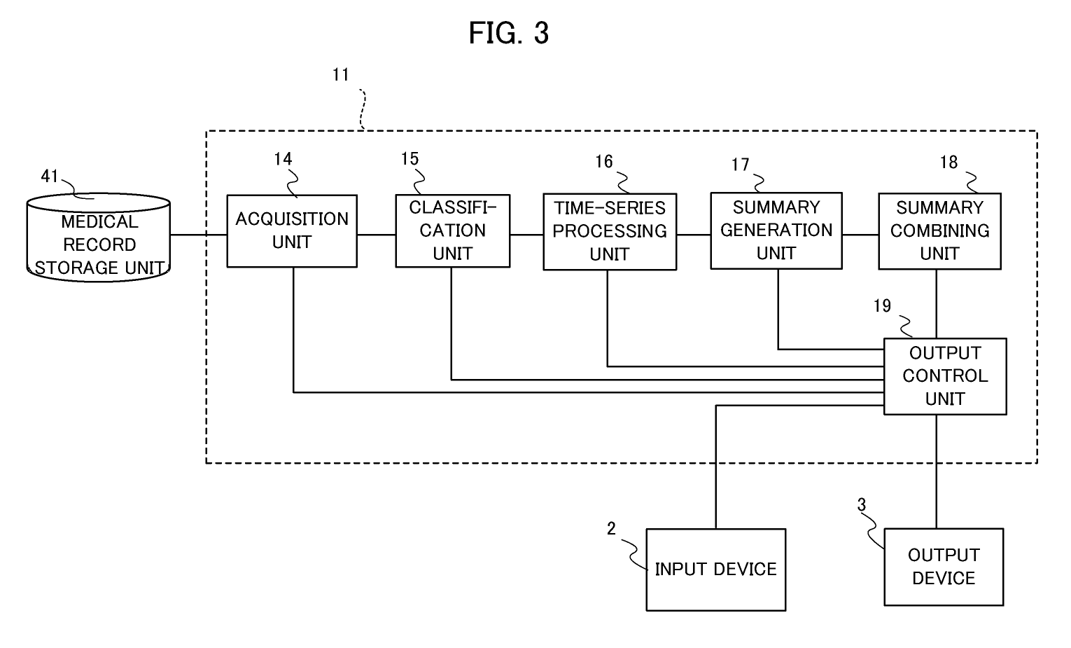

FIG. 3 is an example of functional blocks of a processor 11 of the medical information processing apparatus 1. In terms of functions, the processor 11 of the medical information processing apparatus 1 includes an acquisition unit 14, a classification unit 15, a time-series processing unit 16, a summary generation unit 17, a summary combining unit 18, and an output control unit 19. While blocks that exchange data with each other are connected by a solid line here, a combination of the blocks that exchange data with each other is not limited thereto. The same applies to diagrams of other functional blocks described later.

When detecting the summary generation request according to a user input or the like via the input device 2, the acquisition unit 14 acquires the medical records associated with the patient designated in the summary generation request from the medical record storage unit 41 via the interface 13. In this case, the acquisition unit 14 may extract only a predetermined type of medical records related to a daily life state of the patient from the medical record storage unit 41.

The classification unit 15 classifies the medical records acquired by the acquisition unit 14 into predetermined categories (also referred to as “necessary categories”) related to the daily life state. In this case, the classification unit 15 decomposes each of the medical records for each text (sentence) delimited by a predetermined rule such as a punctuation mark or a line feed, and classifies the decomposed text into the necessary category. In this case, the classification unit 15 may extract a named entity from the medical record, classify the extracted named entity into the necessary category, and then classify the text including the named entity into the necessary category. In this case, any named entity recognition and relational extraction techniques may be used. Note that the classification unit 15 may exclude text that is not classified into any of the necessary categories or a medical record other than text from a target of subsequent processing.

Here, the necessary category is a category related to the daily life state, and is associated with, for example, at least one of a hospital or a clinical department. For example, necessary category information in which at least one of identification information of the hospital or identification information of the clinical department is associated with the necessary category is stored in the storage device 4, the memory 12, or the like. Then, the classification unit 15 refers to the necessary category information and identifies the necessary category based on at least one of the clinical department or the hospital where the target patient receives medical treatment. Examples of the necessary category include movement, living, meals, excretion, bathing/cleaning, putting on and taking off clothes, beauty care, communication, medication management, and sleep. The necessary category is not limited to the above examples, and may be any category representing the daily life state of the patient. Hereinafter, the classification unit 15 classifies the medical records into N (N is an integer of two or more) necessary categories.

The time-series processing unit 16 identifies a chronological order of the medical records classified by the classification unit 15 for each necessary category, and rearranges the medical records in the identified chronological order. For example, the time-series processing unit 16 generates a timeline of the medical records for each necessary category. In this case, the time-series processing unit 16 may identify the related date and time for each text or named entity classified by the classification unit 15, and add date and time information indicating the identified date and time to the text or named entity. At this time, the time-series processing unit 16 may identify date and time information associated with the medical record to which the classified text or named entity belongs as the date and time information related to the classified text or named entity. Note that a method for identifying a time relationship of a named entity (entity) extracted from structured data and unstructured data is disclosed in, for example, JP 2005-508544 A. The classification unit 15 may acquire a named entity associated with date and time information using a language model such as an LLM machine-learned in such a way as to output the related date and time together with the named entity, and the time-series processing unit 16 may identify the chronological order of the medical records for each necessary category with reference to the date and time information.

The summary generation unit 17 generates a summary of the medical records classified for each necessary category. In this case, the summary generation unit 17 generates N summaries related to the N necessary categories. In this case, for example, the summary generation unit 17 generates a prompt for instructing summarization of the medical records classified for each necessary category, and inputs the generated prompt to the LLM to acquire an answer output by the LLM as the summary for each necessary category. In another example, the summary generation unit 17 generates the summary for each necessary category based on a result of text analysis of the medical records classified for each necessary category. Here, examples of the “text analysis” include morphological analysis, syntax analysis, context analysis, time information analysis, factuality analysis, named entity recognition, and semantic analysis. A method for generating the summary based on the result of the text analysis is disclosed in, for example, WO 2024-201577.

In a case where the summary generation unit 17 uses a large language model, if the entire medical record is input as it is, calculation resources (for example, memory usage and inference time) may be excessively consumed. Therefore, the medical information processing apparatus 1 may execute pre-processing of reducing a text amount to be input to the summary generation unit 17. For example, the classification unit 15 or the time-series processing unit 16 may exclude text (a standard header, a signature, an overlapping template sentence, a blank, or meta information that does not need to be presented to a viewer) that does not contribute to summary generation, and shorten the input text to be passed to the summary generation unit 17.

In addition, the medical information processing apparatus 1 may preferentially extract a portion satisfying a predetermined period (for example, the latest N days) or a predetermined condition (for example, a phrase indicating a state change, a numerical change, a call-attention phrase, or the like) from the medical records rearranged in chronological order for each necessary category, and input only the extracted portion to the summary generation unit 17. This makes it possible to hold information necessary for the summary while reducing a token amount to be input.

Further, in a case where the input text exceeds a predetermined maximum input length, the summary generation unit 17 may perform hierarchical summarization processing such as dividing the input text into a plurality of chunks, partially summarizing each of the chunks, and integrating the obtained partial summaries to generate a category summary. At this time, the summary generation unit 17 may suppress redundant output by deleting or integrating overlapping content between the chunks.

As described above, the medical information processing apparatus 1 can reduce calculation resources (for example, memory usage, an operation amount, and inference time) required for language model inference by performing the pre-processing of reducing the text amount (token amount) input to the summary generation unit 17, the extraction based on the period or the condition, and the hierarchical summarization processing. This makes it possible to increase the number of simultaneous processes under the same hardware resources or reduce a response delay.

The summary combining unit 18 generates the summary document obtained by combining the summaries of the respective necessary categories generated by the summary generation unit 17. In this case, the summary combining unit 18 generates one document in which N summaries related to the N necessary categories are connected in such a way that a collection of sentences for each necessary category is not lost. As a result, the summary combining unit 18 can generate an easily readable summary document collected for each necessary category. Note that the summary combining unit 18 may perform processing of arranging a format of the summary document in consideration of readability of the finally generated summary document. For example, in a case where there are two short summaries, the summary combining unit 18 may combine these summaries into one collected sentence.

At this time, preferably, the summary combining unit 18 determines the arrangement of the N summaries in such a way that a longer summary is ranked higher in the generated summary document (that is, set at a position closer to the beginning of the document). In other words, the summary combining unit 18 may arrange a summary of a necessary category having a larger number of characters first. As a result, the summary combining unit 18 can generate a more easily readable summary document.

The output control unit 19 causes the output device 3 to display the summary document generated by the summary combining unit 18, and receives a correction input of the summary document via the input device 2. In this case, for example, the output control unit 19 displays a sentence indicated by the summary document in an input field in which text editing is possible. Then, the output control unit 19 may perform processing of storing the summary document generated by the summary combining unit 18 or the summary document corrected according to the received correction input in the storage device 4, the memory 12, or the like, transmitting the summary document to another device, or printing the summary document by a printer. In addition, the output control unit 19 may cause the output device 3 to display any information generated or acquired by the acquisition unit 14, the classification unit 15, the time-series processing unit 16, the summary generation unit 17, and the summary combining unit 18.

In addition, in a case where the generated summary document is applied to a document (for example, a referral, a medical certificate, a discharge summary, or the like) having a predetermined format, the output control unit 19 may further modify the summary document in such a way that the summary document fits in an entry field for entering the summary document in the document. For example, in a case where the number of characters that can be input to the above entry field is set, the summary document is further summarized in such a way as to be within the number of characters. In this case, the output control unit 19 may further summarize the summary document by using any summarization method (for example, summarization by an LLM). In addition, in a case where there is an entry field for each necessary category, the output control unit 19 may recognize the entry fields of the respective necessary categories and arrange the summaries of the necessary categories generated by the summary generation unit 17 in the related entry fields, respectively.

The output control unit 19 may acquire template information of a document for reference at the time of filling out the summary document in an application target document. Examples of the template information include a document type identifier, an entry field identifier, a display position of the entry field, the maximum number of characters (or the number of lines) of the entry field, a required flag, a correspondence with a category identifier, and an entry order. The template information may be registered in advance in the storage device 4, the memory 12, or the like, or may be acquired from an external device.

The output control unit 19 automatically arranges the summary for each category generated by the summary generation unit 17 in the related entry field based on a correspondence with a category included in the template information. For example, the output control unit 19 may select and arrange a sentence (for example, a sentence including the latest or frequent event in the chronological order) with higher priority from the summaries of the respective categories in accordance with the maximum number of characters for each entry field.

In response to a confirmation operation (for example, a confirmation button 65), the output control unit 19 may generate completed document data in a predetermined data format and register the generated document data in an external device. For example, the output control unit 19 may generate structured data (tagged text or a message in a predetermined format) in which a document type identifier, a patient identifier, an entry field identifier, and a summary text arranged in the entry field are associated with each other, and transmit the structured data to an electronic medical record system or a document management system. As a result, a series of processes from the generation of the summary document to the registration of the document are automated by the apparatus.

In addition, the output control unit 19 may cause the output device 3 to display an input screen for receiving an input of the summary generation request, and may receive the input of the summary generation request on the input screen via the input device 2. In this case, the output control unit 19 supplies the acquisition unit 14 with an input signal input by the input device 2. In addition, the output control unit 19 may receive an input that designates the patient for generating the summary document, the medical record used for the summary document, and the like.

Here, each component of the acquisition unit 14, the classification unit 15, the time-series processing unit 16, the summary generation unit 17, the summary combining unit 18, and the output control unit 19 can be implemented by, for example, the processor 11 executing a program. Each component may also be achieved by recording a necessary program in an optional nonvolatile storage medium and installing the program as necessary. At least a part of these components is not limited to be achieved by software by a program, and may be achieved by a combination of any of hardware, firmware, and software, or the like. At least a part of these components may be achieved using, for example, a user-programmable integrated circuit such as a field-programmable gate array (FPGA) or a microcontroller. In this case, a program including the above components may be achieved by using the integrated circuit. At least a part of the components may include an application specific standard produce (ASSP), an application specific integrated circuit (ASIC), or a quantum processor (quantum computer control chip). In this manner, the components may be achieved by various types of hardware. The same applies to other example embodiments described later. These components may also be achieved by, for example, cooperation of a plurality of computers by using a cloud computing technology or the like.

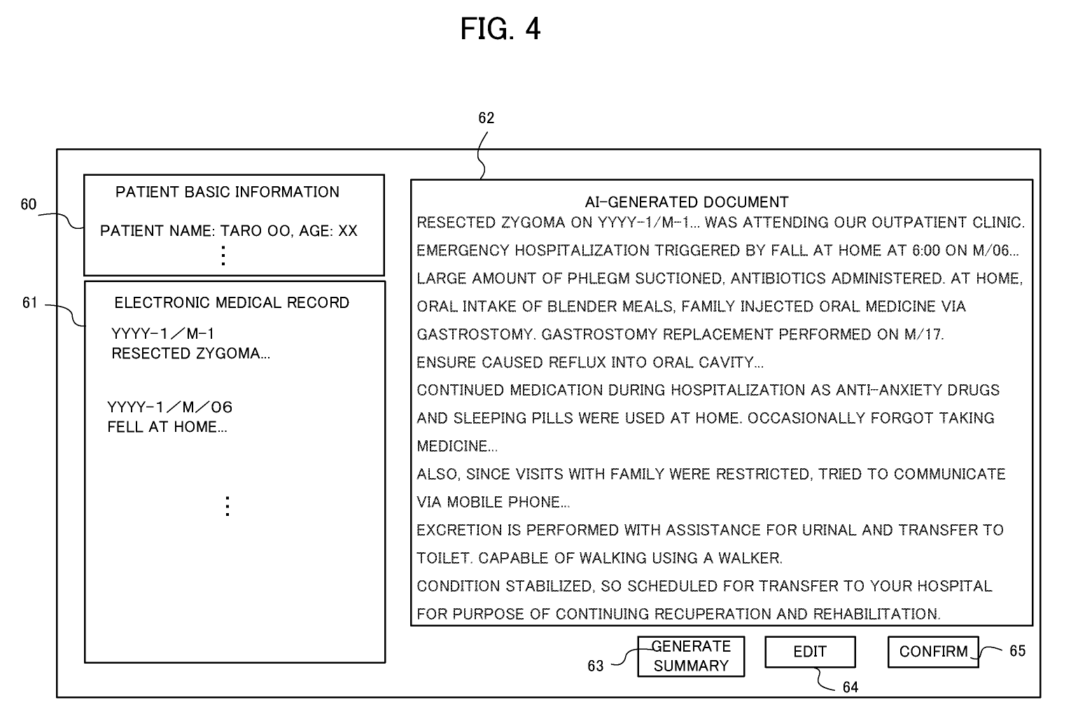

FIG. 4 illustrates a first display example of the summary document displayed on the output device 3 by the output control unit 19. The output control unit 19 supplies a display signal for displaying a display screen illustrated in FIG. 4 to the output device 3, and the output device 3 displays the display screen illustrated in FIG. 4 based on the display signal. Here, for example, the output control unit 19 detects the summary generation request when a summary generation button 63 is selected to instruct generation of a summary document of electronic medical records of patient “Taro ○○”, and displays the summary document of the electronic medical record of the patient “Taro ○○” as an AI-generated document.

In the example illustrated in FIG. 4, the output control unit 19 displays a patient basic information display field 60, an electronic medical record display field 61, an AI-generated document display field 62, the summary generation button 63, an edit button 64, and the confirmation button 65 on the display screen. Here, the output control unit 19 displays basic information (profile and the like) of the patient such as the age, occupation, and the like regarding the patient “Taro ○○” selected by the viewer in the patient basic information display field 60. In addition, the output control unit 19 extracts the electronic medical records which are medical record of the patient “Taro ○○” from the medical record storage unit 41, and displays the extracted electronic medical records in the electronic medical record display field 61.

In addition, the output control unit 19 generates the summary document of the electronic medical records of the patient “Taro ○○” displayed in the electronic medical record display field 61, and displays the generated summary document in the AI-generated document display field 62. In this case, the output control unit 19 summarizes the electronic medical records related to each of N (here, 5) necessary categories related to a clinical department or/and a hospital where the patient “Taro ○○” receives medical treatment, and displays the summary document, obtained by combining the summaries of the respective necessary categories, in the AI-generated document display field 62.

In addition, when detecting that the edit button 64 is selected, the output control unit 19 enables input of text with respect to the AI-generated document display field 62 and receives a correction of the summary document displayed in the AI-generated document display field 62. Further, when detecting that the confirm button 65 is selected, the output control unit 19 confirms the summary document displayed in the AI-generated document display field 62 as the final summary document. In this case, the output control unit 19 may store the finally generated summary document in the storage device 4, the memory 12, or the like, may transmit the summary document to another device, or may print the summary document by a printer. In addition, the output control unit 19 may place the finally generated summary document in an entry field of a document to be applied (a nursing summary or the like), complete the document, and perform storage, transmission, printing, or the like of the completed document (file).

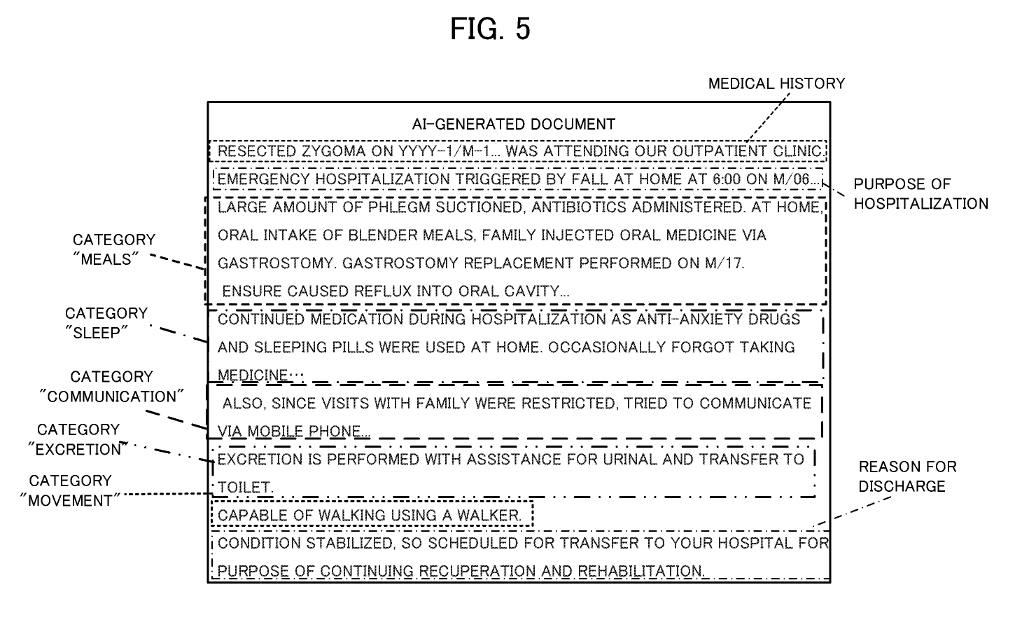

FIG. 5 is an enlarged view in which annotations are added to the summary document displayed in the AI-generated document display field 62. In FIG. 5, a relationship between a sentence constituting the summary document displayed in the AI-generated document display field 62 and a category (including a necessary category) to which the sentence belongs is clearly indicated.

The output control unit 19 identifies “meals”, “sleep”, “communication”, “movement”, and “excretion” as necessary categories, and generates the summary document connecting summaries of the necessary categories. In this manner, one document in which the summaries of the necessary categories are connected is generated in such a way that a collection of sentences for each necessary category is not lost, whereby the output control unit 19 generates an easily readable summary document in which the summaries of the necessary categories are integrated. Further, the summary combining unit 18 determines the arrangement of the summaries of the respective necessary categories in such a way that a longer summary is ranked higher in the generated summary document (that is, set at a position closer to the beginning of the document). Specifically, the summary of the necessary category “meals” having the largest number of characters is arranged at the beginning of the summaries of all the necessary categories, and the summary of the necessary category “movement” having the smallest number of characters is arranged at the end of the summaries of all the necessary categories. As a result, the summary combining unit 18 generates an easily readable summary document.

In addition to the necessary categories, the output control unit 19 classifies texts of the electronic medical records displayed in the electronic medical record display field 61 for each of categories of “medical history”, “purpose of hospitalization”, and “reason for discharge”, and adds summaries of the classified texts for the respective categories to the summary document. In this case, a method for classifying and summarizing the texts for each of the categories of the “medical history”, the “purpose of hospitalization”, and the “reason for discharge” is, for example, the same as the method of classifying and summarizing the text for each necessary category (that is, the processing executed by the classification unit 15, the time-series processing unit 16, and the summary generation unit 17). Then, the output control unit 19 arranges the summary of the “medical history” and the summary of the “purpose of hospitalization” before a group of the summaries of the necessary categories, and arranges the summary of the “reason for discharge” after the group of the summaries of the necessary categories.

In a case where processing of identifying a chronological order of named entities for each necessary category is executed by the time-series processing unit 16, the output control unit 19 may display the named entities arranged in chronological order for each necessary category. In this case, the output control unit 19 may provide tables (that is, a total of N tables) for the necessary categories, respectively, and display a pair of the named entity and the related date and time information side by side for each necessary category.

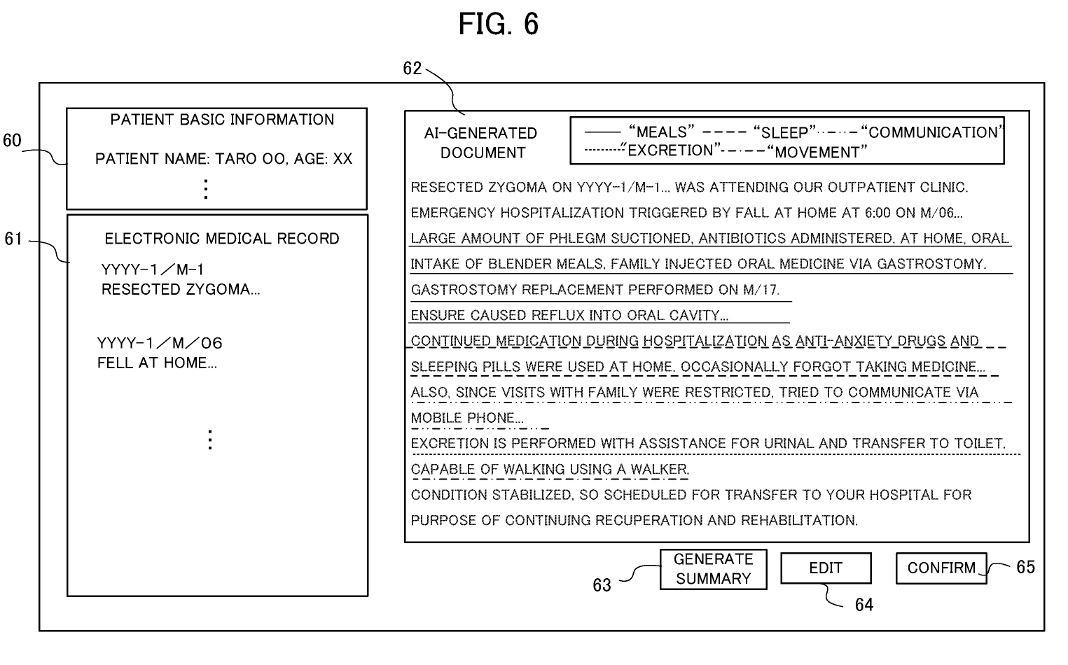

FIG. 6 illustrates a second display example of the summary document displayed on the output device 3 by the output control unit 19. In the second display example, the output control unit 19 underlines a summary portion for each necessary category with a line type associated to the necessary category in such a way that the viewer can identify the summary portion for each necessary category in the AI-generated document display field 62. In this manner, the output control unit 19 displays each of the summaries of the respective necessary categories, displayed as the summary document, in a display mode different for each necessary category. In addition, the output control unit 19 displays the summary document in a mode of clearly indicating a correspondence between each sentence of the summary document and the corresponding necessary category. This enables the viewer to suitably recognize the category to which each sentence of the summary document belongs.

Note that the output control unit 19 may color-code the sentences of the summary document for the necessary categories, respectively, instead of adding the different line type of the underline to the summary document for each necessary category. In addition, the output control unit 19 may display the summary document in any mode in which the viewer can identify the necessary category related to each sentence of the summary document. For example, the output control unit 19 may cause the output device 3 to display the annotated AI-generated document corresponding to FIG. 5 as it is. In addition to the necessary categories, the output control unit 19 may similarly display sentences belonging to the categories of the “medical history”, the “purpose of hospitalization”, and the “reason for discharge” in a mode that allows the viewer to identify correspondences between the sentence and the categories. In addition, when any sentence of the summary document is selected (by mouseover), the output control unit 19 may display a necessary category related to the selected sentence, instead of the display mode of FIG. 6.

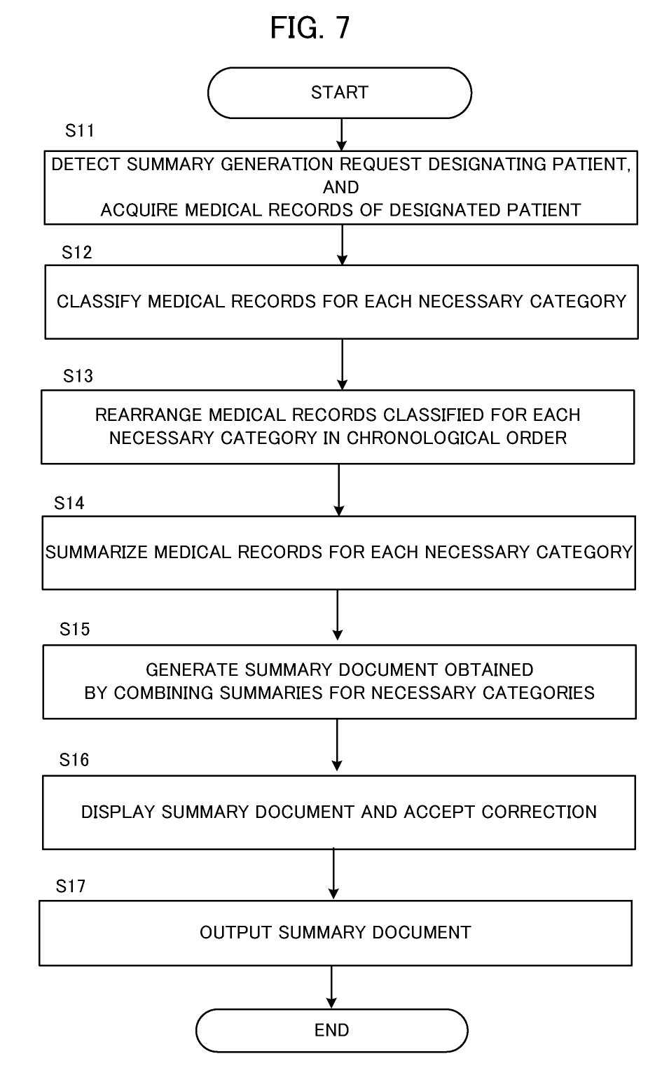

FIG. 7 illustrates an example of a flowchart executed by the medical information processing apparatus 1. For example, the medical information processing apparatus 1 executes processing of the flowchart illustrated in FIG. 7 in a case where a user input requesting generation of a summary document or the like is detected.

First, the medical information processing apparatus 1 detects a summary generation request for medical records designating a patient, and acquires the medical records of the patient designated in the summary generation request (step S11). For example, in a case where a button or the like for instructing summary generation is input on a screen or the like for displaying electronic medical records in a state where the patient is designated, the medical information processing apparatus 1 detects the summary generation request based on an input signal generated by the input device 2 and extracts the medical records of the designated patient from the medical record storage unit 41.

Next, the medical information processing apparatus 1 classifies the medical records acquired in step S11 for each necessary category (step S12). In this case, the medical information processing apparatus 1 may perform processing of identifying the necessary categories related to at least one of a clinical department or a hospital where the designated patient receives medical treatment with reference to necessary category information. Further, the medical information processing apparatus 1 executes processing of rearranging the medical records classified for each necessary category in chronological order (step S13).

Then, the medical information processing apparatus 1 summarizes the medical records classified for each necessary category and arranged in chronological order for each necessary category (step S14). In this case, the medical information processing apparatus 1 generates a total of N summaries related to the N necessary categories, respectively.

Then, the medical information processing apparatus 1 generates a summary document obtained by combining (that is, connecting) the summaries for the necessary categories obtained in step S13 (step S15). Then, the medical information processing apparatus 1 displays the generated summary document and receives an input of a correction (step S16). In this case, the medical information processing apparatus 1 displays the summary document by the output device 3, and receives the input of the correction of the summary document via the input device 2. Then, the medical information processing apparatus 1 outputs the summary document (step S17). In this case, the medical information processing apparatus 1 may modify the summary document in such a way that the finally generated summary document falls within an entry field of a document to be applied. The medical information processing apparatus 1 may store the finally generated summary document in the storage device 4, the memory 12, or the like, may transmit the summary document to another device, or may print the summary document by a printer.

### Second Example Embodiment

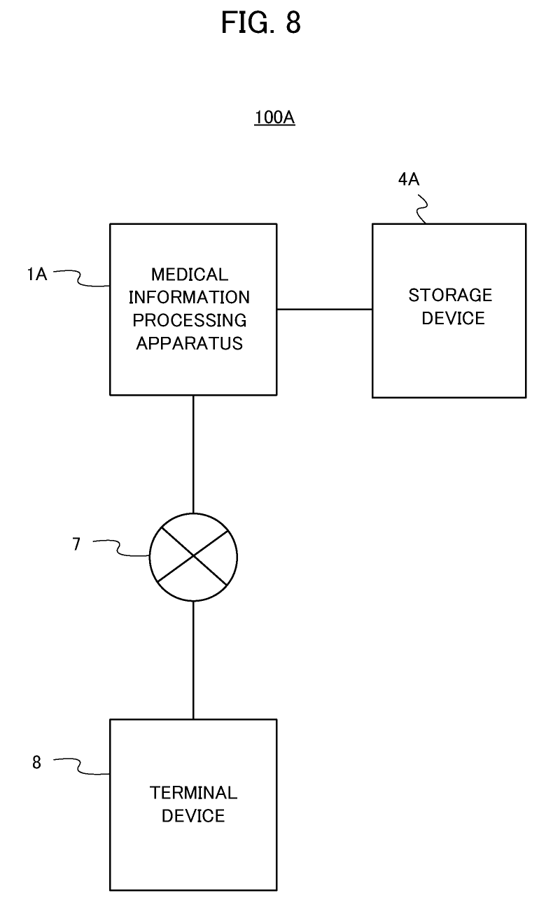

FIG. 8 illustrates a schematic configuration of a medical information processing system 100A. The medical information processing system 100A according to a second example embodiment is a server client model system, and a medical information processing apparatus 1A that functions as a server device performs the processing of the medical information processing apparatus 1 in the first example embodiment. Hereinafter, the same components as those of the first example embodiment are appropriately denoted by the same reference signs, and the description thereof will be omitted.

The medical information processing system 100A mainly includes the medical information processing apparatus 1A that functions as a server, a storage device 4A that stores data similar to the data stored in the storage device 4 according to the first example embodiment, and a terminal device 8 that functions as a client. The medical information processing apparatus 1A and the terminal device 8 perform data communication via a network 7.

The terminal device 8 is a terminal having an input function, a display function, and a communication function, and functions as the input device 2 and the output device 3 illustrated in FIG. 1. The terminal device 8 may be, for example, a personal computer, a tablet terminal, a personal digital assistant (PDA), and the like. The terminal device 8 transmits an input signal or the like based on a user input to the medical information processing apparatus 1A.

The medical information processing apparatus 1A has the same hardware configuration and functional blocks as those of the medical information processing apparatus 1 according to the first example embodiment. Then, the medical information processing apparatus 1A receives the input signal or the like from the terminal device 8 via the network 7, detects a summary generation request for medical records based on the received information, and performs processing such as generation of a summary document based on the summary generation request. The medical information processing apparatus 1A exchanges an output signal indicating information or the like related to a generation result of the summary document and an input signal indicating information or the like related to correction of the generated summary document with the terminal device 8 via the network 7. That is, in this case, the terminal device 8 functions as the input device 2 and the output device 3 in the first example embodiment.

As described above, the medical information processing system 100A according to the second example embodiment can generate the summary document based on the request from the user who handles the terminal device 8 and present the summary document to the user.

### Third Example Embodiment

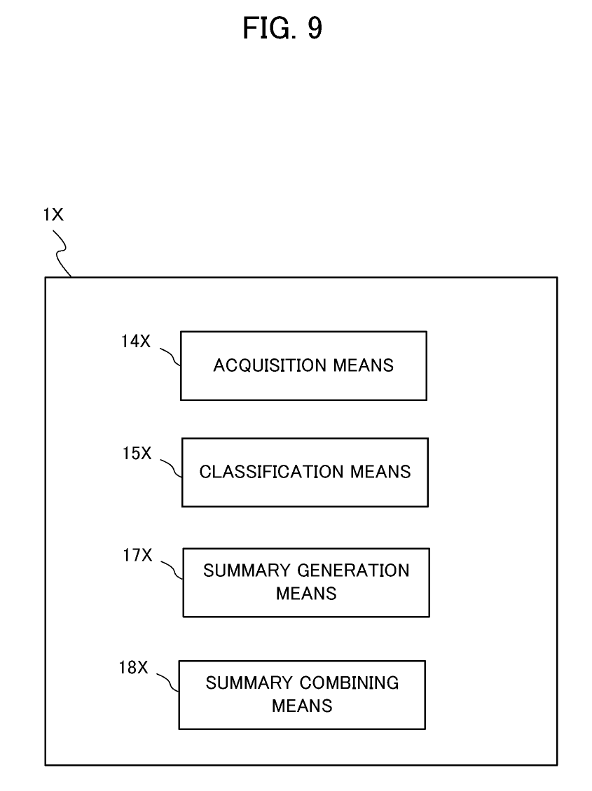

FIG. 9 is a block diagram of a medical information processing apparatus 1X. The medical information processing apparatus 1X includes an acquisition means 14X, a classification means 15X, a summary generation means 17X, and a summary combining means 18X. The medical information processing apparatus 1X may be configured by a plurality of devices.

The acquisition means 14X acquires medical records of a patient. The acquisition means 14X may be, for example, the acquisition unit 14 in the first example embodiment and the second example embodiment. The classification means 15X classifies the medical records for each of categories related to a daily life state. The classification means 15X may be, for example, the classification unit 15 in the first example embodiment and the second example embodiment.

The summary generation means 17X generates, for each of the categories, a summary of the medical records classified for each of the categories. The summary generation means 17X may be, for example, the summary generation unit 17 in the first example embodiment and the second example embodiment. The summary combining means 18X generates a summary document obtained by combining the summaries of the categories. The summary combining means 18X may be, for example, the summary combining unit 18 in the first example embodiment and the second example embodiment.

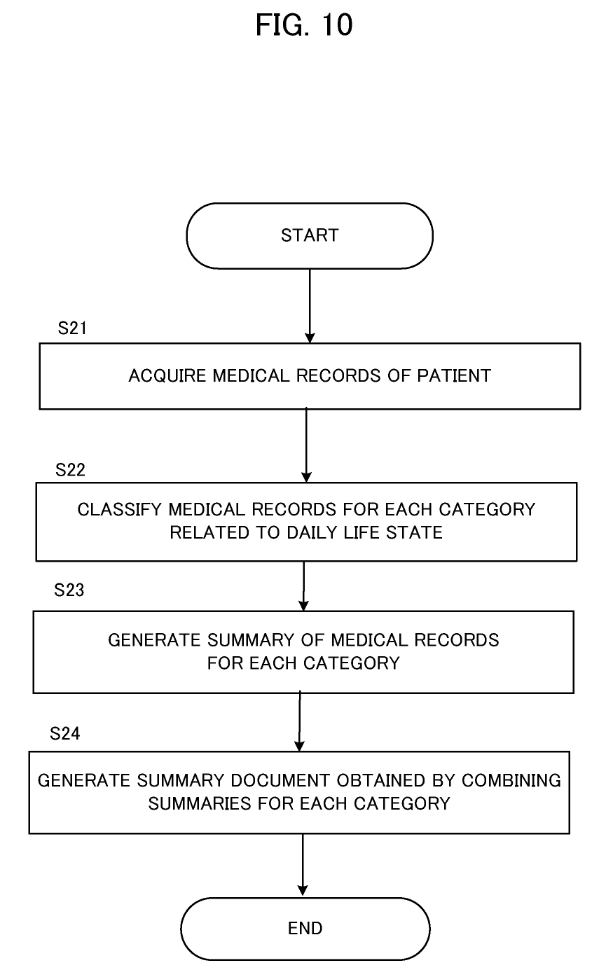

FIG. 10 is an example of a flowchart illustrating a processing procedure executed by the medical information processing apparatus 1X. The acquisition means 14X acquires medical records of a patient (step S21). The classification means 15X classifies the medical records for each of categories related to a daily life state (step S22). The summary generation means 17X generates, for each of the categories, a summary of the medical records classified for each of the categories (step S23). The summary combining means 18X generates a summary document obtained by combining the summaries of the categories (step S24).

According to the third example embodiment, the medical information processing apparatus 1X can suitably generate a summary document regarding a daily life state from medical records.

In the example embodiments described above, the program is stored by any type of a non-transitory computer-readable medium (non-transitory computer readable medium) and can be supplied to a control unit or the like that is a computer. The non-transitory computer-readable medium includes any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

In addition, part or all of the above-described example embodiments (including modifications thereof; the same applies hereinafter) may also be described as the following Supplementary Notes, but the present disclosure is not limited thereto. Further, part or all of the configurations described in the Supplementary Notes dependent on Supplementary Note 1 may also depend on Supplementary Notes 9 and 10 in the same dependent manner as the Supplementary Notes dependent on Supplementary Note 1. Furthermore, without being limited to the apparatuses, methods, and storage media described in the Supplementary Notes, part or all of the configurations described as the Supplementary Notes may similarly be applied, without departing from the scope of the above-described example embodiments, to methods, various types of hardware, software, various recording media (including storage media) for recording software, or systems.

### Supplementary Note 1

A medical information processing apparatus comprising:
an acquisition means for acquiring medical records of a patient;
a classification means for classifying the medical records for each of categories related to a daily life state;
a summary generation means for generating, for each of the categories, a summary of the medical records classified for each of the categories; and
a summary combining means for generating a summary document obtained by combining the summaries of the categories.

### Supplementary Note 2

The medical information processing apparatus according to Supplementary Note 1, further comprising a display control means for displaying the summary document on a display device,
wherein the display control means displays each of the summaries of the categories displayed as the summary document in a display mode different for each of the categories.

### Supplementary Note 3

The medical information processing apparatus according to Supplementary Note 2, wherein the display control means displays the summaries of the categories displayed as the summary document to visually indicate correspondences with the categories, respectively.

### Supplementary Note 4

The medical information processing apparatus according to Supplementary Note 1, wherein the summary combining means generates the summary document by combining the summaries arranged in descending order of a number of characters.

### Supplementary Note 5

The medical information processing apparatus according to Supplementary Note 1, wherein the classification means determines each of the categories based on at least one of a clinical department or a hospital where the patient receives medical treatment.

### Supplementary Note 6

The medical information processing apparatus according to Supplementary Note 1, further comprising a time-series processing means for identifying a chronological order of the medical records classified for each of the categories.

### Supplementary Note 7

The medical information processing apparatus according to Supplementary Note 2, wherein the display control means displays the summary document in a mode of receiving an input for correcting the summary document.

### Supplementary Note 8

The medical information processing apparatus according to Supplementary Note 2, wherein the display control means stores or prints a document in which the summary document is placed in an entry field.

### Supplementary Note 9

A method executed by a computer, comprising:
acquiring medical records of a patient;
classifying the medical records for each of categories related to a daily life state;
generating, for each of the categories, a summary of the medical records classified for each of the categories; and
generating a summary document obtained by combining the summaries of the categories.

### Supplementary Note 10

A non-transitory computer readable storage medium storing a program executed by a computer, the program causing the computer to:
acquire medical records of a patient;
classify the medical records for each of categories related to a daily life state;
generate, for each of the categories, a summary of the medical records classified for each of the categories; and
generate a summary document obtained by combining the summaries of the categories.

While the invention has been particularly shown and described with reference to example embodiments thereof, the invention is not limited to these example embodiments. It will be understood by those of ordinary skill in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. Each example embodiment can be appropriately combined with other example embodiments. All Patent and Non-Patent Literatures mentioned in this specification are incorporated by reference in its entirety.

### DESCRIPTION OF REFERENCE NUMERALS

1, 1A, 1X Medical information processing apparatus

2 Input device

3 Output device

4 Storage device

8 Terminal device

100, 100A Medical information processing system

1. A medical information processing apparatus comprising:
at least one memory configured to store instructions; and
at least one processor configured to execute the instructions to:
acquire medical records of a patient;
classify the medical records for each of categories related to a daily life state;
generate, for each of the categories, a summary of the medical records classified for each of the categories; and
generate a summary document obtained by combining the summaries of the categories.

2. The medical information processing apparatus according to claim 1,
wherein the at least one processor is configured to further execute the instructions to
display the summary document on a display device such that each of the summaries of the categories is displayed in a display mode different for each of the categories.

3. The medical information processing apparatus according to claim 2,
wherein the at least one processor is configured to execute the instructions to display the summary document to visually indicate correspondences between the summaries of the categories and the categories.

4. The medical information processing apparatus according to claim 1,
wherein the at least one processor is configured to execute the instructions to generate the summary document by combining the summaries arranged in descending order of a number of characters.

5. The medical information processing apparatus according to claim 1,
wherein the at least one processor is configured to execute the instructions to determine each of the categories based on at least one of a clinical department or a hospital where the patient receives medical treatment.

6. The medical information processing apparatus according to claim 1,
wherein the at least one processor is configured to further execute the instructions to identify a chronological order of the medical records classified for each of the categories.

7. The medical information processing apparatus according to claim 2,
wherein the at least one processor is configured to execute the instructions to display the summary document in a mode of receiving an input for correcting the summary document.

8. The medical information processing apparatus according to claim 2,
wherein the at least one processor is configured to execute the instructions to store or print a document in which the summary document is placed in an entry field.

9. A method executed by a computer, comprising:
acquiring medical records of a patient;
classifying the medical records for each of categories related to a daily life state;
generating, for each of the categories, a summary of the medical records classified for each of the categories; and
generating a summary document obtained by combining the summaries of the categories.

10. A non-transitory computer readable storage medium storing a program executed by a computer, the program causing the computer to:
acquire medical records of a patient;
classify the medical records for each of categories related to a daily life state;
generate, for each of the categories, a summary of the medical records classified for each of the categories; and
generate a summary document obtained by combining the summaries of the categories.
